(19) United States
(12) Patent Application Publication
(10) Pub. No.: US 2021/0066753 A1
(43) Pub. Date: Mar. 4, 2021

(54) NON-AQUEOUS ELECTROLYTE SECONDARY BATTERY

(71) Applicant: MURATA MANUFACTURING CO., LTD., Kyoto (JP)

(72) Inventors: Masahiro MIYAMOTO, Kyoto (JP); Taichi KOGURE, Kyoto (JP); Masumi FUKUDA, Kyoto (JP)

(21) Appl. No.: 17/096,347

(22) Filed: Nov. 12, 2020

Related U.S. Application Data
(63) Continuation of application No. PCT/JP2019/021594, filed on May 30, 2019.

(30) Foreign Application Priority Data
May 31, 2018 (JP) 2018-105713

(51) Int. Cl. H01M 10/0567; H01M 4/587; H01M 10/0525; C07D 319/06; C07D 493/10; C07D 319/12; C07D 317/40

(52) U.S. Cl. CPC H01M 10/0567; H01M 4/587; H01M 10/0525; H01M 2300/0025; C07D 493/10; C07D 319/12; C07D 317/40; C07D 319/06

(57) ABSTRACT

A non-aqueous electrolyte secondary battery includes a positive electrode, a negative electrode, and an electrolyte. The negative electrode includes an active material and a binder having a mesh structure. The electrolyte includes at least one compound selected from the group consisting of a first cyclic ether having an ether structure at 1st and 3rd positions of a 6- or higher membered ring, a second cyclic ether having an ether structure at 1st and 4th positions of a 6- or higher membered ring, and derivatives of the first cyclic ether and the second cyclic ether.

### CROSS REFERENCE TO RELATED APPLICATIONS

The present application is a continuation of PCT patent application no. PCT/JP2019/021594, filed on May 30, 2019, which claims priority to Japanese patent application no. JP2018-105713 filed on May 31, 2018, the entire contents of which are being incorporated herein by reference.

### BACKGROUND

The present technology generally relates to a non-aqueous electrolyte secondary battery.

In recent years, development has been promoted to increase the charge and discharge capacitance by removing and inserting a greater amount of lithium by the increase in charge pressure of the battery. However, when the charge pressure of the battery is increased, the electrolytic solution is decomposed on the positive electrode side at the time of charge and gas is likely to be generated.

### SUMMARY

The present technology generally relates to a non-aqueous electrolyte secondary battery.

The conventional technology cannot sufficiently suppress the generation of gas at the time of high temperature storage and the increase in internal resistance.

An object of the present invention is to provide a non-aqueous electrolyte secondary battery capable of suppressing gas generation and an increase in internal resistance at the time of high temperature storage.

According to an embodiment of the present technology, a non-aqueous electrolyte secondary battery is provided. The non-aqueous electrolyte secondary battery includes a positive electrode, a negative electrode, and an electrolyte. The negative electrode includes an active material and a binder having a mesh structure and the electrolyte includes at least one compound selected from the group consisting of a first cyclic ether having an ether structure at 1st and 3rd positions of a 6- or higher membered ring, a second cyclic ether having an ether structure at 1st and 4th positions of a 6- or higher membered ring, and derivatives of the first cyclic ether and the second cyclic ether.

According to the present invention, it is possible to suppress gas generation and an increase in internal resistance at the time of high temperature storage.

It should be understood that the effects described here are not necessarily limited and may be any one of the effects described in the present invention or an effect different from them.

### BRIEF DESCRIPTION OF THE FIGURES

FIG. 1 is an exploded perspective view of a non-aqueous electrolyte secondary battery according to an embodiment of the present technology.

FIG. 2 is a sectional view taken along the line II-II in FIG. 1.

FIG. 3 is a sectional SEM image of a negative electrode according to an embodiment of the present technology.

FIG. 4 is a sectional view of a non-aqueous electrolyte secondary battery according to an embodiment of the present technology.

FIG. 5 is an enlarged sectional view illustrating a part of the wound electrode body illustrated in FIG. 4.

FIG. 6 is a block diagram of an electronic device as an application example according to an embodiment of the present technology.

### DETAILED DESCRIPTION

As described herein, the present disclosure will be described based on examples with reference to the drawings, but the present disclosure is not to be considered limited to the examples, and various numerical values and materials in the examples are considered by way of example.

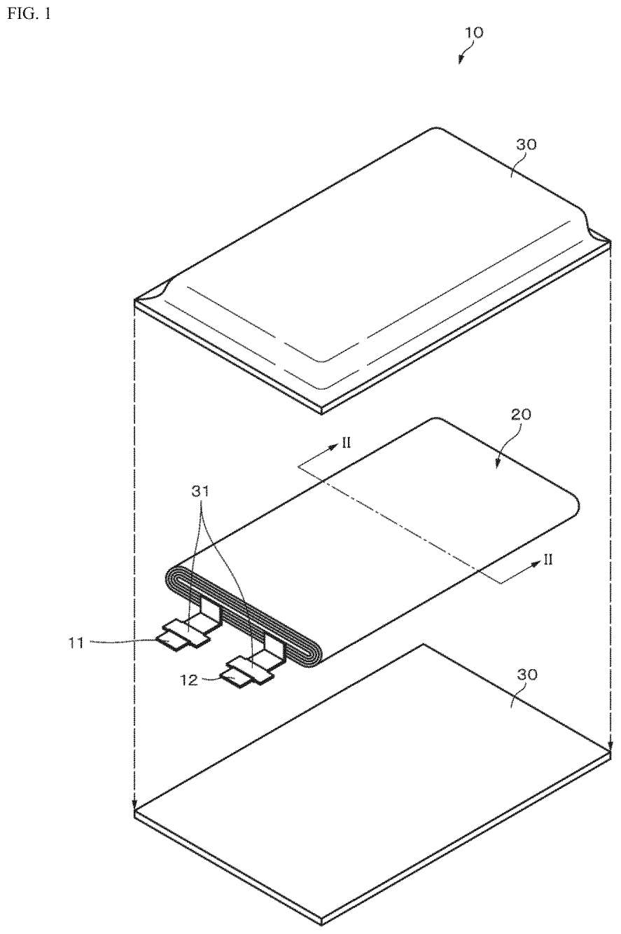

As illustrated in FIG. 1, a non-aqueous electrolyte secondary battery (hereinafter simply referred to as “battery”) 10 according to a first embodiment of the present invention is a so-called laminate type battery, a flat wound electrode body 20 to which a positive electrode lead 11 and a negative electrode lead 12 are attached and an electrolytic solution are housed inside a film-like exterior material 30 in the battery 10, and miniaturization, weight saving, and thinning of the battery 10 are possible. The battery 10 is, for example, a so-called lithium ion secondary battery in which the capacitance of the negative electrode is represented by a capacitance component due to storage and release of lithium that is an electrode reactant.

The positive electrode lead 11 and the negative electrode lead 12 are both led out, for example, in the same direction from the inside to the outside of the exterior material 30. The positive electrode lead 11 is formed of, for example, a metal material such as Al, Ni, or stainless steel or a carbon material. The negative electrode lead 12 is formed of, for example, a metal material such as Ni, Cu, or a composite material thereof. The positive electrode lead 11 and the negative electrode lead 12 have, for example, a thin plate shape or a mesh shape.

The exterior material 30 is formed of, for example, a laminate film exhibiting flexibility. The exterior material 30 has, for example, a configuration in which a heat-sealing resin layer, a metal layer, and a surface protective layer are sequentially laminated. The surface on the heat-sealing resin layer side is the surface on the side on which the wound electrode body 20 is housed. Examples of the material for this heat-sealing resin layer include polypropylene (PP) and polyethylene (PE). Examples of the material for the metal layer include aluminum. Examples of the material for the surface protective layer include nylon (Ny). Specifically, for example, the exterior material 30 is formed of a rectangular aluminum laminate film in which a nylon film, an aluminum foil, and a polyethylene film are bonded to each other in this order. The exterior material 30 is arranged so that, for example, the heat-sealing resin layer side and the wound electrode body 20 face each other, and the respective outer edge portions are in close contact with each other by sealing or an adhesive. A close contact film 31 is inserted between the exterior material 30 and the positive electrode lead 11 and between the exterior material 30 and the negative electrode lead 12 to prevent intrusion of outside air. The close contact film 31 is formed of a material exhibiting close contact property to the positive electrode lead 11 and the negative electrode lead 12, for example, a polyolefin resin such as polyethylene, polypropylene, modified polyethylene, or modified polypropylene.

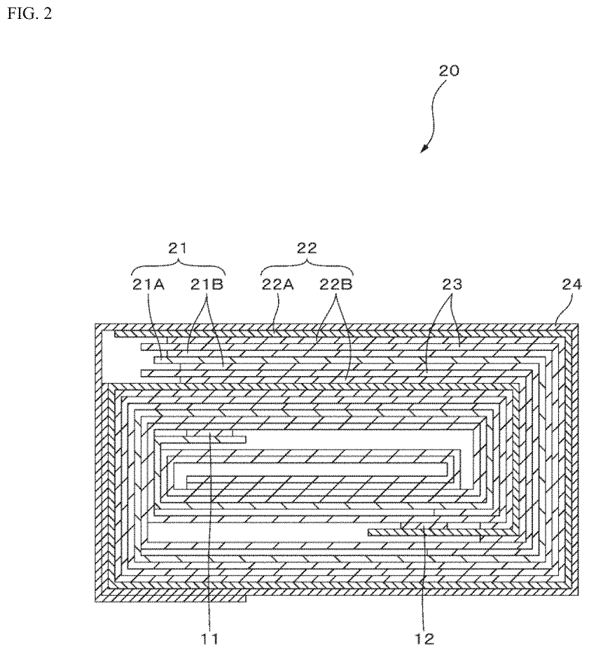

As illustrated in FIG. 2, the wound electrode body 20 as a battery element is obtained by stacking a strip-like positive electrode 21 and a strip-like negative electrode 22 with a strip-like separator 23 interposed therebetween and winding these in a flat and spiral shape, and the outermost peripheral portion thereof is protected by a protective tape 24.

Hereinafter, the positive electrode 21, negative electrode 22, and separator 23 which constitute the wound electrode body 20 will be sequentially described.

The positive electrode 21 includes, for example, a positive electrode current collector 21A and a positive electrode active material layer 21B provided on both surfaces of the positive electrode current collector 21A. The positive electrode current collector 21A is formed of, for example, a metal foil such as an aluminum foil, a nickel foil, or a stainless foil. The positive electrode active material layer 21B contains a positive electrode active material. The positive electrode active material layer 21B may further contain at least one of a binder or a conductive agent, if necessary.

As the positive electrode active material capable of storing and releasing lithium, a lithium-containing compound, for example, lithium oxide, lithium phosphorus oxide, lithium sulfide, or an intercalation compound containing lithium is suitable, and two or more of these may be used in mixture. In order to increase the energy density, a lithium-containing compound which contains lithium, a transition metal element, and oxygen is preferable. Examples of such a lithium-containing compound include a lithium composite oxide having a layered rock salt type structure represented by Formula (A) and a lithium composite phosphate having an olivine type structure represented by Formula (B). The lithium-containing compound is more preferably one containing at least one selected from the group consisting of Co, Ni, Mn, and Fe as a transition metal element. Examples of such a lithium-containing compound include a lithium composite oxide having a layered rock salt type structure represented by Formula (C), Formula (D), or Formula (E), a lithium composite oxide having a spinel type structure represented by Formula (F), or a lithium composite phosphate having an olivine type structure represented by Formula (G). Specific examples thereof include LiNi_{0.50}Co_{0.20}Mn_{0.30}O₂, LiCoO₂, LiNi₂, LiNiₐCO₁₋ₐO₂ (0<a<1), LiMn₂O₄, or LiFePO₄.

LiₚNi_{(i-q-r)}Mn_{q}M1ᵣO_{(2-y)}X_{z} (A)

(In Formula (A), M1 represents at least one selected from the elements belonging to the groups 2 to 15 except Ni and Mn. X represents at least one among the elements belonging to the group 16 and the elements belonging to the group 17 other than oxygen. p, q, y, and z are values within ranges of 0:5≤p≤1.5, 0≤q≤1.0, 0≤r≤1.0, −0.10≤y≤0.20, and 0≤z≤0.2.)

LiₐM2_{b}PO₄ (B)

(In Formula (B), M2 represents at least one selected from the elements belonging to the groups 2 to 15. a and bare values within ranges of 0≤a≤2.0 and 0.5≤b≤2.0.)

Li_{f}Mn_{(i-g-h)}Ni_{g}M3ₕO₍₂₋ⱼ₎Fₖ (C)

(In Formula (C), M3 represents at least one selected from the group consisting of Co, Mg, Al, B, Ti, V, Cr, Fe, Cu, Zn, Zr, Mo, Sn, Ca, Sr, and W. f, g, h, j, and k are values within ranges of 0.8≤f≤1.2, 0<g<0.5, 0≤h≤0.5, g+h<1, −0.1≤j≤0.2, and 0≤k≤0.1. The composition of lithium differs depending on the state of charge and discharge, and the value of f represents a value in the fully discharged state.)

LiₘNi₍₁₋ₙ₎M4ₙO₍₂₋ₚ₎F_{q} (D)

(In Formula (D), M4 represents at least one selected from the group consisting of Co, Mn, Mg, Al, B, Ti, V, Cr, Fe, Cu, Zn, Mo, Sn, Ca, Sr, and W. m, n, p, and q are values within ranges of 0.8≤m≤1.2, 0.005≤n≤0.5, −0.1≤p≤0.2, and 0≤q≤0.1. The composition of lithium differs depending on the state of charge and discharge, and the value of m represents a value in the fully discharged state.)

LiᵣCo₍₁₋ₛ₎M5ₛO₍₂₋ₜ₎Fᵤ (E)

(In Formula (E), M5 represents at least one selected from the group consisting of Ni, Mn, Mg, Al, B, Ti, V, Cr, Fe, Cu, Zn, Mo, Sn, Ca, Sr, and W. r, s, t, and u are values within ranges of 0.8≤r≤1.2, 0≤s<0.5, −0.1≤t≤0.2, and 0≤u≤0.1. The composition of lithium differs depending on the state of charge and discharge, and the value of r represents a value in the fully discharged state.)

LiᵥMn_{2-w}M6_{w}OₓF_{y} (F)

(In Formula (F), M6 represents at least one selected from the group consisting of Co, Ni, Mg, Al, B, Ti, V, Cr, Fe, Cu, Zn, Mo, Sn, Ca, Sr, and W. v, w, x, and y are values within ranges of 0.9≤v≤1.1, 0≤w≤0.6, 3.7≤x≤4.1, and 0≤y≤0.1. The composition of lithium differs depending on the state of charge and discharge, and the value of v represents a value in the fully discharged state.)

Li_{z}M7PO₄ (G)

(In Formula (G), M7 represents at least one selected from the group consisting of Co, Mg, Fe, Ni, Mg, Al, B, Ti, V, Nb, Cu, Zn, Mo, Ca, Sr, W, and Zr. z is a value within a range of 0.9≤z≤1.1. The composition of lithium differs depending on the state of charge and discharge, and the value of z represents a value in the fully discharged state.)

As the positive electrode active material capable of storing and releasing lithium, it is also possible to use inorganic compounds which do not contain lithium such as MnO₂, V₂O₅, V₆O₁₃, NiS, and MoS in addition to these.

The positive electrode active material capable of storing and releasing lithium may be one other than the above. Two or more of the positive electrode active materials exemplified above may be mixed in any combination.

As the binder, for example, at least one selected from the group consisting of resin materials such as polyvinylidene fluoride, polytetrafluoroethylene, polyacrylonitrile, styrene-butadiene rubber, and carboxymethyl cellulose or copolymers containing these resin materials as main components is used.

As the conductive agent, for example, at least one carbon material selected from the group consisting of graphite, carbon fibers, carbon black, Ketjen black, carbon nanotubes and the like is used. The conductive agent may be any material exhibiting conductivity and is not limited to the carbon materials. For example, a metal material or a conductive polymer material may be used as the conductive agent.

The negative electrode 22 includes, for example, a negative electrode current collector 22A and a negative electrode active material layer 22B provided on both surfaces of the negative electrode current collector 22A. The negative electrode current collector 22A is formed of, for example, a metal foil such as a copper foil, a nickel foil, or a stainless foil.

The negative electrode active material layer 22B contains a negative electrode active material. The negative electrode active material layer 22B may further contain at least one of a binder or a conductive agent, if necessary. In this battery 10, it is preferable that the electrochemical equivalent of the negative electrode 22 or the negative electrode active material is greater than the electrochemical equivalent of the positive electrode 21 and lithium metal is not deposited on the negative electrode 22 during charge in theory.

Examples of the negative electrode active material capable of storing and releasing lithium include carbon materials such as non-graphitizable carbon, graphitizable carbon, graphite, pyrolytic carbons, cokes, glassy carbons, organic polymer compound fired bodies, carbon fibers, or activated carbon. Among these, the cokes include pitch coke, needle coke, petroleum coke or the like. The term “organic polymer compound fired bodies” refers to one obtained by firing a polymer material such as phenol resin or furan resin at an appropriate temperature for carbonization, and some organic polymer compound fired bodies are classified as non-graphitizable carbon or graphitizable carbon. These carbon materials are preferable since the change in crystal structure that occurs at the time of charge and discharge is significantly small, a high charge and discharge capacitance can be attained, and favorable cycle characteristics can be attained. Particularly, graphite is preferable since graphite has a great electrochemical equivalent and a high energy density can be attained. Non-graphitizable carbon is preferable since excellent cycle characteristics can be attained.

Furthermore, those having a low charge and discharge potential, specifically those having a charge and discharge potential close to that of lithium metal are preferable since it is possible to easily realize a high energy density of the battery 10.

The graphite may be either of natural graphite or artificial graphite, but artificial graphite is preferable. The intensity ratio (I(002)/I(110)) of the X-ray diffraction intensity I(002) of the (002) plane of artificial graphite to the X-ray diffraction intensity I(110) of the (110) plane of artificial graphite is 500 or more, and thus it is possible to confirm whether or not the graphite is artificial graphite by examining the intensity ratio (I(002)/I(110)). The intensity ratio (I(002)/I(110)) is measured as follows. The diffraction peaks of the (002) plane and (110) plane of graphite were measured using an X-ray diffractometer, and the intensity ratio (I(002)/I(110)) was determined from the top intensity of each peak. In the measurement of X-ray diffraction, the X-ray source was CuKα ray/40 KV/20 mA and the step width was 0.02°.

Other negative electrode active materials capable of increasing the capacitance also include materials containing at least one of a metal element or a metalloid element as a constituent element (for example, an alloy, a compound, or a mixture). This is because a high energy density can be attained when such a material is used. In particular, it is more preferable to use these materials together with the carbon materials since it is possible to attain a high energy density and excellent cycle characteristics. In the present invention, the alloy also includes alloys containing one or more metal elements and one or more metalloid elements in addition to alloys composed of two or more metal elements. The alloy may contain a nonmetallic element. The texture thereof includes a solid solution, a eutectic (eutectic mixture), an intermetallic compound, or coexistence of two or more thereof.

Examples of such a negative electrode active material include a metal element or metalloid element capable of forming an alloy with lithium. Specific examples thereof include Mg, B, Al, Ti, Ga, In, Si, Ge, Sn, Pb, Bi, Cd, Ag, Zn, Hf, Zr, Y, Pd, or Pt. These may be crystalline or amorphous.

The negative electrode active material preferably contains a metal element or metalloid element of the group 4B in the short periodic table as a constituent element and more preferably contains at least either of Si or Sn as a constituent element. This is because Si and Sn have a great ability to store and release lithium and a high energy density can be attained. Examples of such a negative electrode active material include a simple substance, an alloy, or a compound of Si, and a simple substance, an alloy, or a compound of Sn, and materials having one or two or more of these at least at a part.

Examples of Si alloys include those containing at least one selected from the group consisting of Sn, Ni, Cu, Fe, Co, Mn, Zn, In, Ag, Ti, Ge, Bi, Sb, Nb, Mo, Al, P, Ga, and Cr as the second constituent element other than Si. Examples of Sn alloys include those containing at least one selected from the group consisting of Si, Ni, Cu, Fe, Co, Mn, Zn, In, Ag, Ti, Ge, Bi, Sb, Nb, Mo, Al, P, Ga, and Cr as the second constituent element other than Sn.

Examples of Sn compounds or Si compounds include those containing O or C as a constituent element. These compounds may contain the above-mentioned second constituent elements.

Among these, the Sn-based negative electrode active material preferably contains Co, Sn, and C as constituent elements and has a low crystalline or amorphous structure.

Examples of other negative electrode active materials also include metal oxides or polymer compounds capable of storing and releasing lithium. Examples of the metal oxides include lithium-titanium oxide containing Li and Ti such as lithium titanate (Li₄Ti₅O₁₂), iron oxide, ruthenium oxide, or molybdenum oxide. Examples of the polymer compounds include polyacetylene, polyaniline, or polypyrrole.

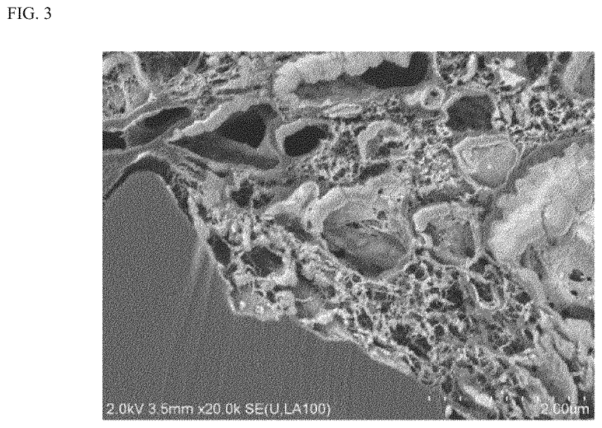

FIG. 3 illustrates a sectional SEM (Scanning Electron Microscope) image of the negative electrode 22. As illustrated in FIG. 3, the binder has a three-dimensional mesh structure (hereinafter, simply referred to as “mesh structure”) and exists in a state in which the mesh structure fills the space between the negative electrode active material grains and the negative electrode active material grains and between the negative electrode active material grains and the negative electrode current collector 22A. Specifically, the binder exists in a state of meshing the space between the negative electrode active material grains and the negative electrode active material grains as well as exists in a state of meshing the space between the negative electrode active material grains and the negative electrode current collector 22A. Hence, the binder contained in the negative electrode active material layer 22B has a structure different from that of a general binder existing so as to cover the surface of the negative electrode active material grains.

The binder having a mesh structure may exist at a part of the entire voids present in the negative electrode active material layer 22B or in the entire voids or substantially the entire voids but preferably exists in the entire voids or substantially the entire voids from the viewpoint of improving the peel strength. Here, the “void” means the space between the negative electrode active material grains and the negative electrode active material grains and between the negative electrode active material grains and the negative electrode current collector 22A.

The binder contains a first binder containing at least one of carboxyalkyl cellulose that is a water-soluble binder or a metal salt thereof and a second binder containing at least one of styrene-butadiene rubber (SBR) that is a rubber-based binder or a derivative thereof. In the first embodiment, a case in which a binder containing the first and second binders is used as the binder will be described, but the binder is not limited to this, and a binder other than the above may be used as long as it is capable of forming a mesh structure.

The carboxyalkyl cellulose includes, for example, at least one of carboxymethyl cellulose (CMC), carboxypropyl methylcellulose, carboxypropyl cellulose, carboxyethyl cellulose, or hydroxypropyl ethylcellulose. The metal constituting the metal salt of carboxyalkyl cellulose includes, for example, at least one selected from the group consisting of Li, Na, K, Rb, Cs, Mg, and Ba.

SBR may contain components other than styrene and butadiene in the molecule. For example, SBR may contain at least one of isoprene or chloroprene in the molecule.

The average pore size of the mesh structure of the binder is preferably 5 nm or more and 5 μm or less, more preferably 100 nm or more and 5 μm or less, still more preferably 1 μm or more and 3 μm or less from the viewpoint of improving the peel strength and the like.

The average pore size of the mesh binder is determined as follows. First, the cross section of the negative electrode 22 is cut out by Focused Ion Beam (FIB) processing or the like, and the image of the cross section is acquired by SEM. At this time, the magnification of the SEM image is set so that the average pore size is sufficiently large. Next, five pores are randomly selected from the acquired SEM image of the cross section, and the width of the pore that is the longest in the straight line distance in each pore is measured as the pore size.

Subsequently, the average pore size is calculated by simply averaging (arithmetic mean) the five measured pore sizes.

The mass ratio (first binder:second binder) of the first binder to the second binder is preferably 1:99 to 90:10, more preferably 1:99 to 40:60, still more preferably 20:80 to 30:70 from the viewpoint of improving the peel strength and the like. The range of each of the above mass ratios shall include the numerical values of the upper limit value and the lower limit value.

The mass ratio of the first binder to the second binder described above is determined by thermogravimetry (TG). Specifically, the mass ratio is determined, for example, by backcalculation from the amount of weight reduced between 300° C. and 390° C. in thermogravimetry.

The mass ratio (binder:active material) of the binder to the negative electrode active material which are contained in the negative electrode active material layer 22B is preferably in a range of 20:80 to 1:99. The range of each of the above mass ratios shall include the numerical values of the upper limit value and the lower limit value. When the proportion of the binder is equal to or less than the mass ratio of 20:80, the increase in internal resistance of the battery 10 can be further suppressed. On the other hand, when the proportion of the binder is equal to or more than the mass ratio of 1:99, the close contact property between the negative electrode active material grains and the negative electrode active material grains and between the negative electrode active material grains and the negative electrode current collector 22A can be further improved. The mass ratio of the binder to the negative electrode active material is determined by thermogravimetry.

The first binder has a viscosity of preferably 10 mPa-s or more and 18000 mPa-s or less, more preferably 100 mPa-s or more and 4000 mPa s or less, still more preferably 1000 mPa s or more and 4000 mPa s or less in the state of an aqueous solution containing the first binder at 1% by mass from the viewpoint of improving the peel strength and the like.

The viscosity of the first binder is determined as follows. First, an aqueous solution (dilute solution) containing CMC at 1% by mass is prepared. Next, the viscosity of the aqueous solution at 25° C. is measured using a B-type viscometer. Specifically, the viscosity of the first bandai is measured using a B-type viscometer as follows. First, a rotor for measurement is arbitrarily selected, and then a container for sample measurement is selected. Next, a fixed amount of the standard solution used for calibrating the viscometer is injected into the prepared rotor and measuring container and subjected to the measurement. The level of rotational speed is changed, and the torque at each rotational speed is measured. The room temperature for measurement and the temperature of the standard solution are both set to 25° C. Then, the apparatus constant is determined by determining the point at which the share rate becomes constant. Next, an aqueous solution in which the first binder is dissolved at 1% by mass is prepared, left to stand at 25° C. for 24 hours, and then subjected to the measurement using the same B-type viscometer and measuring container. The torque is measured while changing the level of the rotational speed, the torque at the same share rate as that when the apparatus constant of the standard solution is determined is measured, and the viscosity of the first binder is determined by multiplying the apparatus constant.

The average grain size of the second binder is preferably 80 nm or more and 500 nm or less, more preferably 100 nm or more and 200 nm or less from the viewpoint of improving the peel strength and the like.

When the second binder is in a dispersion state, the average grain size is determined using a fiber optical dynamic light scattering spectrophotometer (FDLS-3000) manufactured by Otsuka Electronics Co., Ltd. For the measurement, a liquid containing a second binder at a dilution concentration of 0.01% by mass or more and 1% by mass or less is used. In the case of a state in which the second binder is contained in the negative electrode active material layer 22B, the average grain size of the second binder is determined by performing osmium staining, then observing the second binder under SEM, and calculating the average (arithmetic mean) of arbitrary 10 diameters in the image.

Osmium staining is performed as follows. First, osmium tetroxide and the negative electrode 22 are placed in a hermetically sealed box (50° C., 6 hours). Next, ruthenium tetroxide is subjected to the staining treatment (room temperature, 2 hours). Subsequently, cross cushion polishing is performed (5 kV, 8 hours).

The apparatus name of SEM and measurement conditions are presented below.

FE-SEM Hitachi, S-4800 (acceleration voltage: 2 kV), backscattered electron image

The peel strength between the negative electrode active material layer 22B and the negative electrode current collector 22A is preferably 0.1 mN/mm or more and 80 mN/mm or less. When the peel strength is 0.1 mN/mm or more, the cycle characteristics can be further improved. On the other hand, when the peel strength is 80 mN/mm or less, the content of the binder in the negative electrode active material layer 22B can be decreased and thus the increase in internal resistance of the battery 10 can be further suppressed. The peel strength is measured in conformity with iso29862: 2007 (JIS Z 0237).

As the conductive agent, conductive agents similar to those for the positive electrode active material layer 21B can be used.

The separator 23 separates the positive electrode 21 and the negative electrode 22 from each other, prevents short circuit of current due to the contact between both electrodes, and allows lithium ions to pass through. The separator 23 is formed of, for example, a porous film formed of polytetrafluoroethylene, a polyolefin resin (polypropylene (PP), polyethylene (PE) or the like), an acrylic resin, a styrene resin, a polyester resin, a nylon resin, or a resin obtained by blending these resins and may have a structure in which two or more of these porous films are laminated.

Among these, a polyolefin porous film is preferable since this has an excellent short circuit preventing effect and the safety of the battery 10 can be improved by the shutdown effect. Particularly, polyethylene is preferable as a material forming the separator 23 since polyethylene is also excellent in electrochemical stability and a shutdown effect can be attained in a range of 100° C. or more and 160° C. or less. Among these, low-density polyethylene, high-density polyethylene, and linear polyethylene have suitable melting temperatures and are easily procured, and thus are suitably used. In addition, a material obtained by copolymerizing or blending a resin exhibiting chemical stability with polyethylene or polypropylene can be used. Alternatively, the porous film may have a structure composed of three or more layers in which a polypropylene layer, a polyethylene layer, and a polypropylene layer are sequentially laminated. For example, it is desirable to have a three-layer structure of PP/PE/PP and a mass ratio [wt %] of PP to PE in PP:PE=60:40 to 75:25. Alternatively, a single-layer substrate formed of 100 wt % PP or 100 wt % PE can be used from the viewpoint of cost. The method for fabricating the separator 23 may be either of a wet method or a dry method.

A nonwoven fabric may be used as the separator 23. As the fibers constituting the nonwoven fabric, aramid fibers, glass fibers, polyolefin fibers, polyethylene terephthalate (PET) fibers, nylon fibers or the like can be used. A nonwoven fabric may be formed by mixing two or more of these fibers.

The separator 23 may have a configuration including a substrate and a surface layer provided on one surface or both surfaces of the substrate. The surface layer contains inorganic grains exhibiting electrical insulation property and a resin material which binds the inorganic grains to the surface of the substrate and the inorganic grains to each other. This resin material may be, for example, fibrillated and have a three-dimensional network structure in which a plurality of fibrils are linked to each other. The inorganic grains are supported on the resin material having this three-dimensional network structure. The resin material may bind the surface of the substrate and the inorganic grains without being fibrillated. In this case, higher binding property can be attained. By providing the surface layer on one surface or both surfaces of the substrate as described above, the oxidation resistance, heat resistance, and mechanical strength of the separator 23 can be enhanced.

The substrate is a porous film which is permeable to lithium ions and is formed of an insulating film having a predetermined mechanical strength, and it is preferable that the substrate has characteristics to exhibit high resistance to the electrolytic solution, exhibit low reactivity, and hardly expand since the electrolytic solution is retained in the holes of the substrate.

As the material forming the substrate, the resin material or nonwoven fabric forming the above-described separator 23 can be used.

The inorganic grains contain at least one selected from the group consisting of a metal oxide, a metal nitride, a metal carbide, a metal sulfide and the like. As the metal oxide, it is possible to suitably use aluminum oxide (alumina, Al₂O₃), boehmite (hydrated aluminum oxide), magnesium oxide (magnesia, MgO), titanium oxide (titania, TiO₂), zirconium oxide (zirconia, ZrO₂), silicon oxide (silica, SiO₂), yttrium oxide (yttria, Y₂O₃) or the like. As the metal nitride, it is possible to suitably use silicon nitride (Si₃N₄), aluminum nitride (AlN), boron nitride (BN), titanium nitride (TiN) or the like. As the metal carbide, it is possible to suitably use silicon carbide (SiC), boron carbide (B₄C) or the like. As the metal sulfide, it is possible to suitably use barium sulfate (BaSO₄) or the like. Among the above-mentioned metal oxides, it is preferable to use alumina, titania (particularly those having a rutile type structure), silica, or magnesia and it is more preferable to use alumina.

The inorganic grains may contain minerals such as porous aluminosilicate such as zeolite (M_{2/n}O.Al₂O₃.xSiO₂.yH₂O, M is a metal element, x≥2, y≥0), layered silicate, barium titanate (BaTiO₃), or strontium titanate (SrTiO₃). The inorganic grains exhibit oxidation resistance and heat resistance, and the surface layer of the positive electrode-facing side surface containing the inorganic grains exhibits strong resistance to the oxidizing environment in the vicinity of the positive electrode at the time of charge. The shape of the inorganic grains is not particularly limited, and any of spherical, plate-like, fibrous, cubic, or random-shaped inorganic grains can be used.

The grain size of the inorganic grains is preferably in a range of 1 nm or more and 10 μm or less. This is because it is difficult to procure the inorganic grains when the grain size is smaller than 1 nm and the distance between the electrodes is electrodes is far, the amount of active material filled in the limited spaces not sufficiently attained, and the battery capacitance is low when the grain size is larger than 10 m.

Examples of the resin material forming the surface layer include resins exhibiting high heat resistance as at least either of the melting point or the glass transition temperature thereof is 180° C. or more such as fluorine-containing resins such as polyvinylidene fluoride and polytetrafluoroethylene, fluorine-containing rubber such as vinylidene fluoride-tetrafluoroethylene copolymer and ethylene-tetrafluoroethylene copolymer, rubbers such as styrene-butadiene copolymer or hydrides thereof, acrylonitrile-butadiene copolymer or hydrides thereof, acrylonitrile-butadiene-styrene copolymer or hydrides thereof, methacrylic acid ester-acrylic acid ester copolymer, styrene-acrylic acid ester copolymer, acrylonitrile-acrylic acid ester copolymer, ethylene propylene rubber, polyvinyl alcohol, and polyvinyl acetate, cellulose derivatives such as ethyl cellulose, methyl cellulose, hydroxyethyl cellulose, and carboxymethyl cellulose, polyphenylene ether, polysulfone, polyether sulfone, polyphenylene sulfide, polyetherimide, polyimide, polyamide such as wholly aromatic polyamide (aramid), polyamide-imide, polyacrylonitrile, polyvinyl alcohol, polyether, an acrylic acid resin, or polyester. These resin materials may be used singly or in mixture of two or more thereof. Among these, a fluorine-based resin such as polyvinylidene fluoride is preferable from the viewpoint of oxidation resistance and flexibility and it is preferable to contain aramid or polyamide-imide from the viewpoint of heat resistance.

As the method for forming the surface layer, it is possible to use, for example, a method in which a slurry containing a matrix resin, a solvent, and inorganic grains is applied onto a substrate (porous film) and the applied slurry is allowed to pass through a poor solvent of the matrix resin and a bath of a good solvent of the solvent for phase separation and then dried.

The above-described inorganic grains may be contained in the porous film as a substrate. The surface layer may not contain inorganic grains but may be formed only of a resin material.

The electrolytic solution, which is a liquid electrolyte, is a so-called non-aqueous electrolytic solution and contains a non-aqueous solvent, an electrolyte salt, and a first additive, and the electrolyte salt is dissolved in the non-aqueous solvent. The electrolytic solution preferably further contains a second additive from the viewpoint of suppressing gas generation and an increase in internal resistance at the time of high temperature storage. An electrolyte layer containing an electrolytic solution and a polymer compound serving as a retainer for retaining this electrolytic solution may be used instead of the electrolytic solution. In this case, the electrolyte layer may be in a gel form.

Examples of the non-aqueous solvent include ethylene carbonate (EC), propylene carbonate (PC), butylene carbonate (BC), dimethyl carbonate (DMC), diethyl carbonate (DEC), and ethyl methyl carbonate (EMC) as carbonic acid esters, methyl acetate (MA), ethyl acetate (EA), propyl acetate (PA), butyl acetate (BA), methyl propionate (MP), ethyl propionate (EP), propyl propionate (PP), and butyl propionate (BP) as carboxylic acid esters, and γ-butyrolactone and γ-valerolactone as lactone-based ones. These may be used singly or in mixture of a plurality thereof.

The electrolyte salt contains, for example, at least one of light metal salts such as a lithium salt. Examples of the lithium salt include lithium hexafluorophosphate (LiPF₆), lithium tetrafluoroborate (LiBF₄), lithium perchlorate (LiClO₄), lithium hexafluoroarsenate (LiAsF₆), lithium tetraphenylborate (LiB(C₆H₅)₄), lithium methanesulfonate (LiCH₃SO₃), lithium trifluoromethanesulfonate (LiCF₃SO₃), lithium tetrachloroaluminate (LiAlCl₄), dilithium hexafluorosilicate (Li₂SiF₆), lithium chloride (LiCl), and lithium bromide (LiBr).

The first additive is for forming a solid electrolyte interphase (SEI) on the positive electrode 21. The first additive contains at least one compound (hereinafter referred to as “cyclic ether-based compound”) selected from the group consisting of a first cyclic ether having an ether structure at the 1st and 3rd positions, a second cyclic ether having an ether structure at the 1st and 4th positions, and derivatives thereof and preferably contains at least one selected from the group consisting of the first cyclic ether and a derivative thereof from the viewpoint of suppressing gas generation and an increase in internal resistance at the time of high temperature storage. The first additive may further contain a known additive capable of forming a solid electrolyte interphase on the positive electrode 21 together with the cyclic ether-based compound.

The first and second cyclic ethers are each independently cyclic ethers of a 6- or higher membered ring, preferably a 6- or higher membered ring and an 8- or lower membered ring. When the first and second cyclic ethers are a 6- or higher membered ring, a favorable solid electrolyte interphase can be formed on the positive electrode 21. On the other hand, the reason why it is preferable to set the first and second cyclic ethers to 8- or lower membered rings is that the first and second cyclic ethers are less likely to be decomposed and the effect as an additive is extremely low when the number of carbon atoms constituting the first and second cyclic ethers increases.

The first cyclic ether having an ether structure at the 1st and 3rd positions is preferably 1,3-dioxane. The second cyclic ether having an ether structure at the 1st and 4th positions is preferably 1,4-dioxane. The derivative of the first cyclic ether is preferably a derivative of 1,3-dioxane. The derivative of the second cyclic ether is preferably a derivative of 1,4-dioxane.

The derivative of 1,3-dioxane preferably includes at least one of derivatives of 1,3-dioxane represented by the following Formulas (1) and (2).

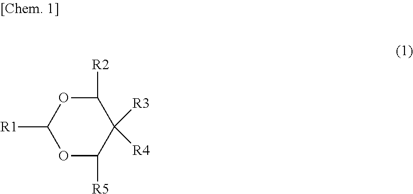

(Where R1 to R5 are each independently a hydrogen group, a hydrocarbon group which may have a substituent (excluding those containing oxygen or nitrogen), or a substituent containing nitrogen or oxygen. R1 to R5 may be bonded to each other. At least one of R1 to R5 is a hydrocarbon group which may have a substituent (excluding those containing oxygen or nitrogen) or a substituent containing nitrogen or oxygen, preferably a substituent containing nitrogen or oxygen.)

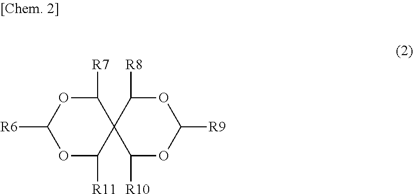

(Where R6 to R11 are each independently a hydrogen group, a hydrocarbon group which may have a substituent (excluding those containing oxygen or nitrogen), or a substituent containing nitrogen or oxygen. At least one of R6 to R11 is a hydrocarbon group which may have a substituent (excluding those containing oxygen or nitrogen) or a substituent containing nitrogen or oxygen, preferably a substituent containing nitrogen or oxygen.)

The derivative of 1,4-dioxane preferably includes at least one of derivatives of 1,4-dioxane represented by the following Formulas (3), (4), and (5).

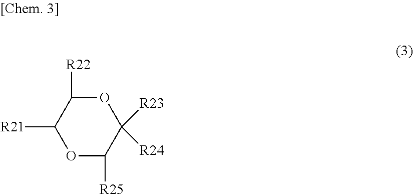

(Where R21 to R25 are each independently a hydrogen group, a hydrocarbon group which may have a substituent (excluding those containing oxygen or nitrogen), or a substituent containing nitrogen or oxygen. R21 to R25 may be bonded to each other. At least one of R21 to 2R5 is a hydrocarbon group which may have a substituent (excluding those containing oxygen or nitrogen) or a substituent containing nitrogen or oxygen, preferably a substituent containing nitrogen or oxygen.)

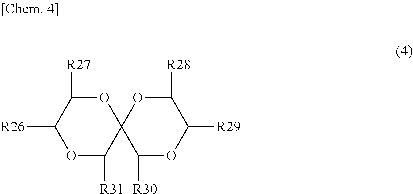

(Where R26 to R31 are each independently a hydrogen group, a hydrocarbon group which may have a substituent (excluding those containing oxygen or nitrogen), or a substituent containing nitrogen or oxygen. At least one of R26 to R31 is a hydrocarbon group which may have a substituent (excluding those containing oxygen or nitrogen) or a substituent containing nitrogen or oxygen, preferably a substituent containing nitrogen or oxygen.)

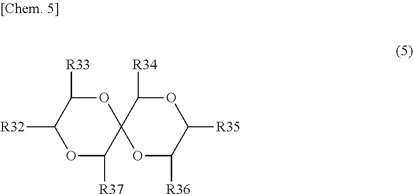

(Where R32 to R37 are each independently a hydrogen group, a hydrocarbon group which may have a substituent (excluding those containing oxygen or nitrogen), or a substituent containing nitrogen or oxygen. At least one of R32 to R37 is a hydrocarbon group which may have a substituent (excluding those containing oxygen or nitrogen) or a substituent containing nitrogen or oxygen, preferably a substituent containing nitrogen or oxygen.)

Examples of the hydrocarbon group which may have a substituent (excluding those containing oxygen or nitrogen) in Formulas (1) to (5) include an aliphatic hydrocarbon group such as an alkyl group, a hydrocarbon group such as an aromatic hydrocarbon group, or one obtained by substituting hydrogen groups of these with a substituent (excluding those containing oxygen and nitrogen). The aliphatic hydrocarbon group may be linear, branched, or cyclic. Specific examples of the substituent containing nitrogen include an amino group, an amide group, an imide group, a cyano group (nitrile group), an isonitrile group, an isoimide group, an isocyanate group, an imino group, a nitro group, a nitroso group, a pyridine group, a triazine group, a guanidine group, or an azo group, or groups having these groups such as a hydrocarbon groups having these groups. Examples of the hydrocarbon group include an aliphatic hydrocarbon group such as an alkyl group and an aromatic hydrocarbon group. The aliphatic hydrocarbon group may be linear, branched, or cyclic. The aliphatic hydrocarbon group may be a tertiary, secondary, or primary aliphatic hydrocarbon group. The number of carbon atoms in the substituent containing nitrogen is not particularly limited but is, for example, preferably 0 or more and 6 or less. Examples of the substituent containing oxygen include a hydroxyl group, an ether group, an ester group, an aldehyde group, a peroxide group, or a carbonate group or groups having these groups such as hydrocarbon groups having these groups. The number of carbon atoms in the substituent containing oxygen is not particularly limited but is, for example, preferably 0 or more and 6 or less. Examples of the hydrocarbon group include an aliphatic hydrocarbon group such as an alkyl group and an aromatic hydrocarbon group. The aliphatic hydrocarbon group may be linear, branched, or cyclic. The aliphatic hydrocarbon group may be a tertiary, secondary, or primary aliphatic hydrocarbon group. A hydrocarbon group which may have a substituent (excluding those containing oxygen or nitrogen) and a substituent containing nitrogen or oxygen is, for example, a monovalent group.

The content of the cyclic ether-based compound is preferably 0.1% by mass or more and 1% by mass or less with respect to the entire mass of the electrolytic solution. When the content of the cyclic ether-based compound is 0.1% by mass or more, gas generation at the time of high temperature storage can be significantly suppressed. On the other hand, when the content of the cyclic ether-based compound is 1% by mass or less, the amount of gas generated at the time of high temperature storage can be significantly decreased, an increase in resistance can be significantly suppressed, and particularly a favorable gas generation suppressing effect and load characteristics can be both achieved.

The content of the cyclic ether-based compound can be determined, for example, as follows. First, the battery 10 is disassembled in an inert atmosphere such as a glove box, and the components of electrolytic solution are extracted using DMC, a heavy solvent, and the like. Next, the content of the cyclic ether-based compound in the electrolytic solution is determined by subjecting the obtained extract to GC-MS (Gas Chromatograph-Mass Spectrometry) measurement.

Specific examples of the derivatives of 1,3-dioxane include 4-methyl-1,3-dioxane, 2,4-dimethyl-1,3-dioxane, 4-phenyl-1,3-dioxane, and 3,9-divinyl-2,4,8,10-tetraoxaspiro[5.5]undecane, but the derivatives of 1,3-dioxane are not limited to these. These may be used singly or in mixture of a plurality thereof.

Specific examples of the derivatives of 1,4-dioxane include 1,4-dioxane-2-one and 2,5-bis[(acetoxymerclio)methyl]-1,4-dioxane, but the derivatives of 1,4-dioxane are not limited to these. These may be used singly or in mixture of a plurality thereof.

Examples of known additives which can be used together with the cyclic ether-based compounds include at least one of a dinitrile compound or a cyclic disulfonic anhydride.

Examples of the dinitrile compound include succinonitrile, glutaronitrile, adiponitrile, pimeronitrile, suberonitrile, azeranitrile, sebaconitrile, phthalonitrile, 3,3′-oxydipropionitrile, ethylene glycol bis(propionitrile)ether, and 3,3′-thiodipropionitrile. These may be used singly or in mixture of a plurality thereof.

The content of the dinitrile compound is preferably 3% by mass or more and 7% by mass or less with respect to the entire mass of the electrolytic solution. When the content of the dinitrile compound is 3% by mass or more, the amount of gas generated at the time of high temperature storage can be effectively suppressed. On the other hand, when the content of the dinitrile compound is 7% by mass or less, the amount of gas generated at the time of high temperature storage can be sufficiently suppressed, the load characteristics and cycle characteristics can be maintained, and the functions of dinitrile compound can be particularly exerted. The content of dinitrile compound is measured in the same manner as the content of cyclic ether-based compound described above.

Examples of the cyclic disulfonic anhydride include 1,2-ethanedisulfonic anhydride, 1,2-benzenedisulfonic anhydride, and 1,3-propanedisulfonic anhydride. These may be used singly or in mixture of a plurality thereof.

The content of cyclic disulfonic anhydride is preferably 0.1% by mass or more and 0.8% by mass or less with respect to the entire mass of the electrolytic solution. When the content of cyclic disulfonic anhydride is 0.1% by mass or more, a general side reaction of solvent due to the interface protection of the positive electrode 21 can be suppressed. On the other hand, when the content of cyclic disulfonic anhydride is 0.8% by mass or less, it is possible to decrease the resistance while suppressing the side reaction at the interface of the positive electrode 21 and it is possible to suppress the side reaction and decrease the resistance at the same time. When the content exceeds 0.8% by mass, excessive solid electrolyte interphase formation on the positive electrode 21 side may cause deterioration in load characteristics due to an increase in resistance. The content of cyclic disulfonic anhydride is measured in the same manner as the content of cyclic ether-based compound described above.

The second additive is for forming a solid electrolyte interphase (SEI) on the negative electrode 22. The second additive is preferably a compound having LUMO energy of 0.60 eV or less. When a compound having such LUMO energy is used, a favorable solid electrolyte interphase can be formed on the negative electrode 22. Hence, it is possible to suppress gas generation and an increase in internal resistance at the time of high temperature storage. When the LUMO energy is 0.60 eV or less as described above, the second additive is reduced and decomposed earlier than a general non-aqueous solvent, and a solid electrolyte interphase can be formed on the negative electrode 22. Hence, the decomposition of general non-aqueous solvent can be suppressed. The lower limit value of LUMO energy is preferably −0.10 eV or more from the viewpoint of the energy level that does not inhibit Li insertion into the negative electrode 22. LUMO energy is determined by molecular orbital calculation by calculation level: B3LYP/6-3IG (d,p) and software: Gaussina 09.

The second additive preferably contains at least one of a compound represented by the following Formula (6) or a compound represented by Formula (7). As a solid electrolyte interphase derived from at least one of these compounds is formed on the negative electrode 22 by charge and discharge, it is possible to further improve gas generation and an increase in internal resistance at the time of high temperature storage.

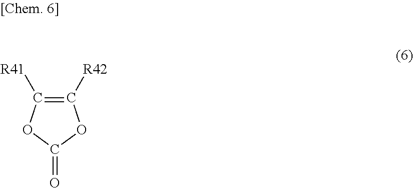

(Where R41 and R42 are each independent a hydrogen group or an alkyl group)

The compound represented by Formula (6) is a vinylene carbonate-based compound. Examples of this vinylene carbonate-based compound include vinylene carbonate (1,3-dioxol-2-one) (LUMO: −0.02 eV), methyl vinylene carbonate (4-methyl-1,3-dioxol-2-one), ethyl vinylene carbonate (4-ethyl-1,3-dioxol-2-one), 4,5-dimethyl-1,3-dioxol-2-one, and 4,5-diethyl-1,3-dioxol-2-one. These may be used singly or in mixture of a plurality thereof. Among these, vinylene carbonate is preferable. This is because vinylene carbonate can be easily procured and a high effect is attained.

The content of the compound represented by Formula (6) is preferably 0.1% by mass or more and 0.5% by mass or less with respect to the entire mass of the electrolytic solution. The compound represented by Formula (6) mainly has an interface protecting function on the negative electrode 22 side and has an interface protecting function for both the positive electrode 21 and the negative electrode 22 in a high charge pressure region in which the fully charged state of the battery 10 is 4.45 V. When the content of the compound represented by Formula (6) is 0.1% by mass or more, the interface side reaction of both the positive electrode 21 and the negative electrode 22 can be particularly suppressed in a high charge pressure region in which the fully charged state of the battery 10 is 4.45 V. On the other hand, when the content of the compound represented by Formula (6) is 0.5% by mass or less, it is possible to decrease the resistance while suppressing the interface side reaction and it is possible to suppress the side reaction and decrease the resistance at the same time. When the content exceeds 0.5% by mass, excessive solid electrolyte interphase formation on the positive electrode 21 side may cause deterioration in load characteristics due to an increase in resistance. The content of the compound represented by Formula (6) is measured in the same manner as the content of the cyclic ether-based compound described above.

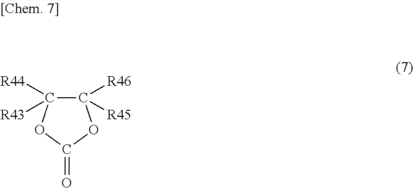

(Where R43 to R46 are each independently a hydrogen group, a halogen group, an alkyl group, or an alkyl halide group. At least one of R23 to R26 is a halogen group or an alkyl halide group). The halogen group is preferably a fluorine group. The alkyl halide group is preferably an alkyl fluoride group.)

Examples of the compound represented by Formula (7) include 4-fluoro-1,3-dioxolane-2-one (LUMO: +0.52 eV), 4-chloro-1,3-dioxolane-2-one, 4,5-difluoro-1,3-dioxolane-2-one, tetrafluoro-1,3-dioxolan-2-one, 4-chloro-5-fluoro-1,3-dioxolan-2-one, 4,5-dichloro-1,3-oxolan-2-one, tetrachloro-1,3-dioxolane-2-one, 4,5-bistrifluoromethyl-1,3-dioxolan-2-one, 4-trifluoromethyl-1,3-dioxolan-2-one, 4,5-difluoro-4,5-dimethyl-1,3-dioxolane-2-one, 4,4-difluoro-5-methyl-1,3-dioxolan-2-one, 4-ethyl-5,5-difluoro-1,3-dioxolan-2-one, 4-fluoro-5-trifluoromethyl-1,3-dioxolane-2-one, 4-methyl-5-trifluoromethyl-1,3-dioxolane-2-one, 4-fluoro-4,5-dimethyl-1,3-dioxolan-2-one, 5-(1,1-difluoroethyl)-4,4-difluoro-1,3-dioxolane-2-one, 4,5-dichloro-4,5-dimethyl-1,3-dioxolane-2-one, 4-ethyl-5-fluoro-1,3-dioxolan-2-one, 4-ethyl-4,5-difluoro-1,3-dioxolane-2-one, 4-ethyl-4,5,5-trifluoro-1,3-dioxolan-2-one, and 4-fluoro-4-methyl-1,3-dioxolan-2-one. These may be used singly or in mixture of a plurality thereof.

Among these, 4-fluoro-1,3-dioxolan-2-one or 4,5-difluoro-1,3-dioxolan-2-one is preferable. This is because vinylene carbonate can be easily procured and a high effect is attained.

The content of the compound represented by Formula (7) is preferably 1% by mass or more and 7% by mass or less with respect to the entire mass of the electrolytic solution. When the content of the compound represented by Formula (7) is 1% by mass or more, the maintenance factor of cycle characteristics can be improved. On the other hand, when the content of the compound represented by Formula (7) is 7% by mass or less, it is possible to particularly improve the maintenance factor of cycle characteristics and it is also possible to particularly suppress the amount of gas generated at the time of high temperature storage. When the content exceeds 7% by mass, it may be difficult to suppress gas generation at the time of high temperature storage with the mesh structure of the binder contained in the negative electrode 22 and the first additive exhibiting the gas suppressing function. The content of the compound represented by Formula (7) is measured in the same manner as the content of the cyclic ether-based compound described above.

The second additive may contain at least one of a lithium salt, a carboxylic anhydride, or a disulfonic anhydride instead of at least one of the compound represented by Formula (6) or the compound represented by Formula (7), or together with at least one of the compound represented by Formula (6) or the compound represented by Formula (7). However, it is preferable to contain at least either of the compound represented by Formula (6) or the compound represented by Formula (7) from the viewpoint of suppressing gas generation and an increase in internal resistance at the time of high temperature storage.

Examples of the lithium salt include lithium salts having an oxalic acid skeleton such as at least one of lithium bis(oxalate)borate (LiBOB), lithium fluoro(oxalate)borate (LiFOB), lithium difluoro(oxalate)borate (LiDFOB), lithium tetrafluoro(oxalate)phosphate (LiTFOP), or lithium difluorobis(oxalate)phosphate (LiDFOP).

Examples of the carboxylic anhydride include at least one of succinic anhydride, phthalic anhydride, glutaric anhydride, or maleic anhydride.

In the battery 10 having the above-described configuration, when charge is performed, for example, lithium ions are released from the positive electrode active material layer 21B and stored in the negative electrode active material layer 22B via the electrolytic solution. When discharge is performed, for example, lithium ions are released from the negative electrode active material layer 22B and stored in the positive electrode active material layer 21B via the electrolytic solution.

Next, an example of the method for manufacturing the battery 10 according to the first embodiment of the present invention will be described.

The positive electrode 21 is fabricated as follows. First, for example, a positive electrode active material, a conductive agent, and a binder are mixed together to prepare a positive electrode mixture, and this positive electrode mixture is dispersed in a solvent such as N-methyl-2-pyrrolidone (NMP) to prepare a paste-like positive electrode mixture slurry. Next, this positive electrode mixture slurry is applied to the positive electrode current collector 21A, the solvent is dried, compression molding is performed using a roll pressing machine or the like to form the positive electrode active material layer 21B, and the positive electrode 21 is thus fabricated.

The negative electrode 22 is fabricated by either of the first or second fabricating step presented below. The step for fabricating the negative electrode may be any one by which a mesh structure can be imparted to the binder and is not limited to these first and second fabricating steps.

First, for example, a negative electrode active material, a first binder, and a second binder are mixed together to prepare a negative electrode mixture, and this negative electrode mixture is dispersed in water as a solvent to prepare a paste-like negative electrode mixture slurry. Next, the prepared negative electrode mixture slurry is applied to the negative electrode current collector 22A while containing bubbles in the negative electrode mixture slurry and applying ultrasonic waves to the negative electrode mixture slurry.

The gas constituting the bubbles includes, for example, at least one of nitrogen, oxygen, argon, hydrogen, helium, air, carbonic acid gas, acetylene, propane, or carbon dioxide. Carbon dioxide may be in a solid state (namely, dry ice). The frequency of ultrasonic waves is, for example, in a range of 20 kHz or more and 3 mHz or less.

The size of the pore size in the finally obtained mesh structure can be controlled by setting the size of the bubbles contained in the negative electrode mixture slurry. The size of the bubbles can be changed by the frequency of ultrasonic waves, and the bubbles tend to be smaller as the frequency is higher. It is also possible to control the pore size in the mesh structure by adjusting the slurry viscosity with the molecular weight of the first binder used and the amount of water, but the range of pore size controlled by adjusting the slurry viscosity is not as wide as the range of pore size controlled by adjusting the bubble size. Hence, it is preferable that the bubble size is taken as the main control factor and the slurry viscosity is taken as the auxiliary control factor.

Subsequently, the negative electrode mixture slurry which is applied and contains bubbles is dried to form a negative electrode active material layer 22B containing a binder having a mesh structure on the negative electrode current collector 22A. Thereafter, the negative electrode active material layer 22B is compression-molded by a roll pressing machine or the like to fabricate the negative electrode 22.

First, a paste-like negative electrode mixture slurry is prepared in the same manner as in the first fabricating step. Next, the prepared negative electrode mixture slurry is applied to the negative electrode current collector 22A, and the applied negative electrode mixture slurry is rapidly frozen and then dried in a vacuum state. The negative electrode active material layer 22B containing a binder having a mesh structure is thus formed on the negative electrode current collector 22A. Thereafter, the negative electrode active material layer 22B is compression-molded by a roll pressing machine or the like to fabricate the negative electrode 22.

The freezing temperature for rapid freezing is, for example, in a range of −80° C. or more and −20° C. or less. The degree of vacuum in the vacuum state is, for example, in a range of 20 torr or less. By adjusting the amount of water blended in the negative electrode mixture slurry, it is possible to control the size of the pore size in the mesh structure. Specifically, as the amount of water blended increases, the viscosity of the negative electrode mixture slurry decreases, the number of bubbles increases, and thus the pore size in the finally obtained mesh structure increases. The size of the pore size can be controlled to some extent by adjusting the molecular weight (viscosity) and the degree of etherification of the first binder.

The wound electrode body 20 is fabricated as follows. First, the positive electrode lead 11 is attached to the end portion of the positive electrode current collector 21A by welding and the negative electrode lead 12 is attached to the end portion of the negative electrode current collector 22A by welding. Next, the positive electrode 21 and negative electrode 22 are stacked with the separator 23 interposed therebetween to form a stacked body, and then this stacked body is wound in its longitudinal direction, and the protective tape 24 is pasted to the outermost peripheral portion to fabricate the wound electrode body 20. A predetermined electrolytic solution is injected into the wound electrode body 20.

The wound electrode body 20 is sealed with the exterior material 30 as follows. First, for example, the wound electrode body 20 is sandwiched between the flexible exterior material 30, and the outer edge portions of the exterior material 30 are brought into close contact with each other and sealed by heat seal or the like. At that time, the close contact film 31 is inserted between the positive electrode lead 11 and the exterior material 30 and between the negative electrode lead 12 and the exterior material 30. The close contact film 31 may be attached to each of the positive electrode lead 11 and the negative electrode lead 12 in advance. The exterior material 30 may be embossed in advance to form a concave portion as housing space for housing the wound electrode body 20. As described above, the battery 10 in which the wound electrode body 20 is housed in the exterior material 30 is obtained. Next, the battery 10 may be molded by a heat press if necessary.

In the battery 10 according to the first embodiment, the negative electrode 22 contains an active material and a binder having a mesh structure and the electrolytic solution contains at least one selected from the group consisting of a first cyclic ether having an ether structure at the 1st and 3rd positions of a 6- or higher membered ring, a second cyclic ether having an ether structure at the 1st and 4th positions of a 6- or higher membered ring, and derivatives of these, and thus it is possible to suppress gas generation at the time of high temperature storage and swelling of the battery 10 at the time of high temperature storage. It is also possible to suppress an increase in internal resistance of the battery 10 at the time of high temperature storage.

As the negative electrode 22 contains a binder having a mesh structure, the active sites of the negative electrode active material increase, and thus the second additive can be efficiently reacted on the negative electrode 22 side. Hence, a favorable solid electrolyte interphase can be formed on the negative electrode 22. Consequently, it is also possible to suppress an increase in internal resistance of the battery 10 at the time of high temperature storage.

As the second additive can be efficiently reacted on the negative electrode 22 side, it is possible to suppress the reaction of the first additive on the negative electrode 22 side. Hence, in the battery 10 in which the negative electrode 22 contains a binder having a mesh structure, the first additive can be efficiently reacted on the positive electrode side, and the amount of the second additive added can be decreased as compared with a battery containing a binder not having a mesh structure in the negative electrode. Consequently, a favorable solid electrolyte interphase can be formed on the positive electrode 21 only by adding a small amount of the second additive (for example, 0.1% by mass or more and 1% by mass or less with respect to the entire mass of the electrolytic solution), and it is thus possible to suppress an increase in interfacial resistance on the surface of the positive electrode 21.

When a binder having a mesh structure and artificial graphite as a negative electrode active material are used in combination, the effect of suppressing gas generation and an increase in internal resistance at the time of high temperature storage is particularly remarkably exerted. The specific surface area of artificial graphite is smaller than the specific surface area of natural graphite. For this reason, by increasing the active sites of the negative electrode active material with a binder having a mesh structure, the effect of efficiently reacting the second additive on the negative electrode 22 side is remarkably exerted in artificial graphite as compared with natural graphite.

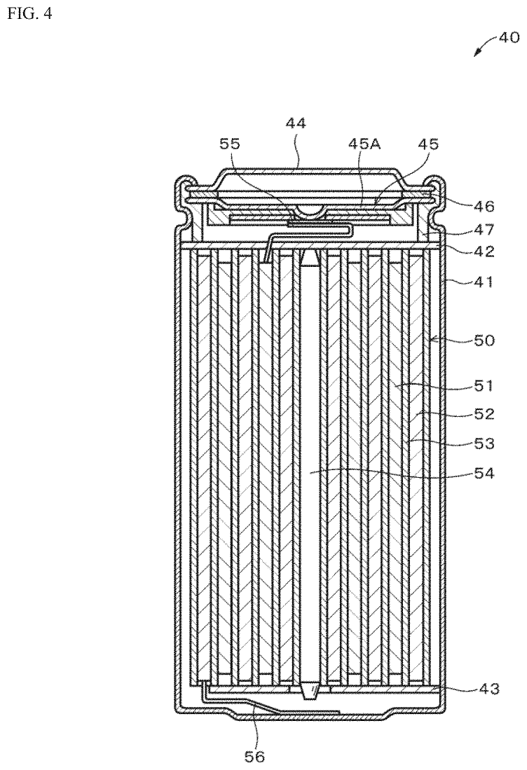

As illustrated in FIG. 4, a battery 40 according to a second embodiment of the present invention is a so-called cylindrical type and includes a wound electrode body 20 in which a pair of strip-like positive electrode 51 and strip-like negative electrode 52 are stacked with a separator 53 interposed therebetween and then wound inside a substantially hollow columnar battery can (exterior material) 41. The battery can 41 is formed of nickel-plated iron, aluminum or the like and has one end portion closed and the other end portion open. An electrolytic solution as a liquid electrolyte is injected into the battery can 41, and the positive electrode 51, the negative electrode 52, and the separator 53 are impregnated with the electrolytic solution. A pair of insulating plates 42 and 43 is disposed perpendicularly to the wound peripheral surface so as to sandwich the wound electrode body 50 therebetween. The electrolytic solution is similar to the electrolytic solution in the first embodiment.

A battery lid 44 and a safety valve mechanism 45 and a positive temperature coefficient element (PTC element) 46 which are provided inside this battery lid 44 are attached to the open end portion of the battery can 41 by being crimped with a sealing gasket 47 interposed therebetween. The inside of the battery can 41 is thus hermitically sealed. The battery lid 44 is formed of, for example, a material similar to that of the battery can 41. The safety valve mechanism 45 is electrically connected to the battery lid 44 and is configured so that a disk plate 45A is inverted to disconnect the electrical connection between the battery lid 44 and the wound electrode body 50 when the internal pressure of the battery 40 is equal to or higher than a certain level by an internal short circuit, heating from the outside, or the like. The sealing gasket 47 is formed of, for example, an insulating material, and its surface is coated with asphalt.

For example, a center pin 54 is inserted in the center of the wound electrode body 50. A positive electrode lead 55 formed of aluminum or the like is connected to the positive electrode 51 of the wound electrode body 50, and a negative electrode lead 56 formed of nickel or the like is connected to the negative electrode 52. The positive electrode lead 55 is electrically connected to the battery lid 44 by being welded to the safety valve mechanism 45, and the negative electrode lead 56 is welded and electrically connected to the battery can 41.

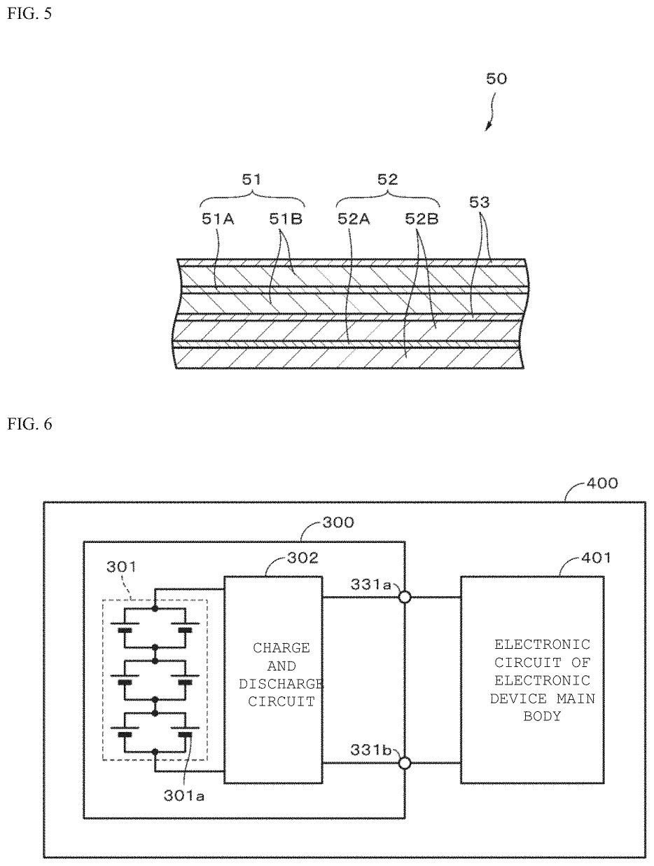

As illustrated in FIG. 5, the positive electrode 51 includes a positive electrode current collector 51A and a positive electrode active material layer 51B provided on both surfaces of the positive electrode current collector 51A. The negative electrode 52 includes a negative electrode current collector 52A and a negative electrode active material layer 52B provided on both surfaces of the negative electrode current collector 52A. The configurations of the positive electrode current collector 51A, the positive electrode active material layer 51B, the negative electrode current collector 52A, the negative electrode active material layer 52B, and the separator 53 are similar to those of the positive electrode current collector 21A, the positive electrode active material layer 21B, the negative electrode current collector 22A, the negative electrode active material layer 22B, and the separator 23 in the first embodiment, respectively.

In the battery 40 according to the second embodiment, gas generation at the time of high temperature storage can be suppressed, and thus the operation of the safety valve can be suppressed. It is also possible to suppress an increase in internal resistance of the battery 10 at the time of high temperature storage.

In a third embodiment, an electronic device which includes the battery 10 according to the first embodiment or the battery 40 according to the second embodiment described above will be described.

As illustrated in FIG. 6, an electronic device 400 according to the third embodiment of the present invention includes an electronic circuit 401 of the electronic device main body and a battery pack 300. The battery pack 300 is electrically connected to the electronic circuit 401 via a positive electrode terminal 331a and a negative electrode terminal 331b. The electronic device 400 has, for example, a configuration in which the battery pack 300 is freely attached and detached.

Examples of the electronic device 400 include laptop personal computers, tablet computers, mobile phones (for example, smartphones), personal digital assistants (PDA), display devices (Liquid Crystal Display (LCD), Electro Luminescence (EL) display, electronic paper and the like), imaging devices (for example, digital still cameras, digital video cameras and the like), audio devices (for example, portable audio players), game consoles, cordless phones, e-books, electronic dictionaries, radios, headphones, navigation systems, memory cards, pacemakers, hearing aids, electric power tools, electric shavers, refrigerators, air conditioners, TVs, stereos, water heaters, microwave ovens, dishwashers, washing machines, dryers, lighting equipment, toys, medical equipment, robots, road conditioners, and traffic lights, but the electronic device 400 is not limited thereto.

The electronic circuit 401 includes, for example, a Central Processing Unit (CPU), a peripheral logic unit, an interface unit, a storage unit, and the like and controls the entire electronic device 400.

The battery pack 300 includes an assembled battery 301 and a charge and discharge circuit 302. The battery pack 300 may further include an exterior material (not illustrated) which houses the assembled battery 301 and the charge and discharge circuit 302, if necessary.

The assembled battery 301 is configured by connecting a plurality of secondary batteries 301a in series and/or in parallel. The plurality of secondary batteries 301a are connected, for example, n in parallel and m in series (n and m are positive integers). FIG. 6 illustrates an example in which six secondary batteries 301a are connected two in parallel and three in series (2P3S). As the secondary battery 301a, the battery according to the second or third embodiment described above is used.

Here, a case in which the battery pack 300 includes the assembled battery 301 including the plurality of secondary batteries 301a is described, but a configuration in which the battery pack 300 includes one secondary battery 301a instead of the assembled battery 301 may be adopted.

The charge and discharge circuit 302 is a control unit which controls charge and discharge of the assembled battery 301. Specifically, the charge and discharge circuit 302 controls charge of the assembled battery 301 at the time of charge. On the other hand, the charge and discharge circuit 302 controls discharge of the electronic device 400 at the time of discharge (that is, when the electronic device 400 is used).

As the exterior material, for example, a case formed of a metal, a polymer resin, or a composite material thereof can be used. Examples of the composite material include a laminated body in which a metal layer and a polymer resin layer are laminated.

### EXAMPLES

Hereinafter, the present invention will be specifically described with reference to Examples, but the present invention is not limited only to these Examples.

### Example 1

The positive electrode was fabricated as follows. First, lithium carbonate (Li₂CO₃) and cobalt carbonate (CoCO₃) are mixed together at a molar ratio of 0.5:1 and then fired in air at 900° C. for 5 hours to obtain lithium-cobalt composite oxide (LiCoO₂) as a positive electrode active material. Next, a positive electrode mixture was obtained by mixing 97 parts by mass of lithium-cobalt composite oxide obtained as described above, 1 part by mass of carbon black as a conductive agent, and 2 parts by mass of polyvinylidene fluoride (PVdF) as a binder and then dispersed in N-methyl-2-pyrrolidone to obtain a paste-like positive electrode mixture slurry. Next, the positive electrode mixture slurry was applied to both surfaces of a positive electrode current collector formed of a strip-like aluminum foil (12 μm thick), dried, and then compression-molded using a roll pressing machine to form a positive electrode active material layer.

The negative electrode was fabricated as follows. First, 97 parts by mass of graphite powder as a negative electrode active material and 3 parts by mass of a binder were mixed together to prepare a negative electrode mixture. As the binder, one obtained by mixing CMC (first binder) and SBR (second binder) at a mass ratio of CMC:SBR=1:1 was used. Next, the negative electrode mixture was dispersed in water as a solvent to prepare a paste-like negative electrode mixture slurry. Next, the prepared negative electrode mixture slurry was applied to one surface of a strip-like copper foil (negative electrode current collector) while containing air in the form of bubbles in the negative electrode mixture slurry and applying ultrasonic waves to the negative electrode mixture slurry. At this time, the frequency of ultrasonic waves was set to 500 kHz. Thereafter, the applied negative electrode mixture slurry was dried and then compression-molded using a roll pressing machine to form a negative electrode active material layer.

The electrolytic solution was prepared as follows. First, ethylene carbonate (EC), propylene carbonate (PC), diethyl carbonate (DEC), and propyl propionate were mixed together at a mass ratio of EC:PC:DEC:propyl propionate=20:10:30:40 to prepare a mixed solvent. Subsequently, an electrolytic solution was prepared by dissolving lithium hexafluorophosphate (LiPF₆) as an electrolyte salt in this mixed solvent so as to have a concentration of 1 mol/kg. Next, the first additive (1,3-dioxane) was added to the electrolytic solution so that the content of the first additive was the value (by mass) presented in Table 1 with respect to the entire mass of the electrolytic solution.

A laminate type battery was fabricated as follows. First, an aluminum positive electrode lead was welded to the positive electrode current collector, and a copper negative electrode lead was welded to the negative electrode current collector. Subsequently, the positive electrode and the negative electrode were brought into close contact with each other with a microporous polyethylene film interposed therebetween and then wound in the longitudinal direction, and a protective tape was attached to the outermost peripheral portion to fabricate a flat-shaped wound electrode body.

Next, this wound electrode body was loaded between the exterior materials, and three sides of the exterior materials were heat-sealed, and one side was not heat-sealed but was open. As the exterior material, a moisture proof aluminum laminate film in which a 25 μm thick nylon film, a 40 μm thick aluminum foil, and a 30 μm thick polypropylene film were laminated in this order from the outermost layer was used. Thereafter, the electrolytic solution was injected through the opening of the exterior material, and the remaining one side of the exterior material was heat-sealed under reduced pressure to hermetically seal the wound electrode body. The intended laminate type battery was thus obtained.

### Examples 2 to 7

Laminate type batteries were obtained in the same manner as in Example 1 except that the first additive (1,3-dioxane, 4-methyl-1,3-dioxane, 3,9-divinyl-2,4,8,10-tetraoxaspiro[5.5]undecane, 1,4-dioxane) and the second additive (vinylene carbonate, 4-fluoro-1,3-dioxolane-2-one) were added to the electrolytic solution so that the contents of the first additive and second additive were the values (% by mass) presented in Table 1 with respect to the entire mass of the electrolytic solution.

### Comparative Example 1

The negative electrode mixture slurry prepared without containing bubbles in the slurry and without applying ultrasonic waves to the slurry was applied to both surfaces of a strip-like copper foil as it was. The electrolytic solution was used without adding both the first additive and the second additive to the electrolytic solution. A laminate type battery was obtained in the same manner as in Example 1 except for these matters.

### Comparative Example 2

A laminate type battery was obtained in the same manner as in Example 1 except that the electrolytic solution was used without adding both the first additive and the second additive to the electrolytic solution.

### Comparative Examples 3 and 5

The negative electrode mixture slurry prepared without containing bubbles in the slurry and without applying ultrasonic waves to the slurry was applied to both surfaces of a strip-like copper foil as it was. Only the second additive was added to the electrolytic solution.

Laminate type batteries were obtained in the same manner as in Examples 2 and 3 except for these matters.

### Comparative Examples 4 and 6

Laminate type batteries were obtained in the same manner as in Examples 2 and 3 except that only the second additive was added to the electrolytic solution.

### Comparative Example 7

A laminate type battery was obtained in the same manner as in Example 1 except that the negative electrode mixture slurry prepared without containing bubbles in the slurry and without applying ultrasonic waves to the slurry was applied to both surfaces of a strip-like copper foil as it was.

First, the battery was charged and discharged two cycles in an atmosphere of 23° C. and then charged in the same atmosphere at a constant current density of 0.7 C until the battery voltage reached 4.45 V. Subsequently, the battery was charged at a constant voltage of 4.45 V until the current density reached 0.05 C, and then the thickness D₀ of the battery was measured. Next, the battery was stored in a constant temperature bath at 60° C. for 720 hours, and then the thickness D₁ of the battery was measured. Finally, the swelling rate of the battery after high temperature storage was calculated by the following equation.

Swelling rate of battery after high temperature storage [%]=[(D₁−D₀)/D₀]×100

“0.7 C” is a current value at which the battery capacitance (theoretical capacitance) is fully discharged in 10/7 hours, and “0.05 C” is a current value at which the battery capacitance is fully discharged in 20 hours.

The battery stored in a constant temperature bath at 60° C. for 720 hours was taken out from the bath, charged and discharged two cycles in an atmosphere of 23° C., and then charged in the same atmosphere at a constant current density of 0.7 C until the battery voltage reached 4.45 V. Subsequently, the battery was charged at a constant voltage of 4.45 V until the current density reached 0.05 C, and then the 1 Hz resistance value R₁ illustrated in the Nyquist plot and Bode plot using the AC impedance method were measured. Finally, the resistance change rate of the battery after high temperature storage was calculated by the following equation.

Resistance change rate after high temperature storage [%]=[(R₁−R₀)/R₀]×100

The resistance value R₀ is a 1 Hz resistance value measured by the same procedure as above using the battery before being subjected to high temperature storage.

TABLE 1

| | Presence | | | | | | | | |
|---|---|---|---|---|---|---|---|---|---|
| | or absence | First additive | | | | Second | | Swelling | |
| | of mesh | 1,3- | 4-methyl- | Spiro | 1,4- | additive | | by storage | Resistance |
| | structure | DOX | 1.3-DOX | compound | DOX | VC | FEC | at 60° C. | change |
| | of negative | (% by | (% by | (% by | (% by | (% by | (% by | for 720 | rate after |
| | electrode | mass) | mass) | mass) | mass) | mass) | mass) | hours (%) | storage (%) |
| Example 1 | Presence | 1 | 0 | 0 | 0 | 0 | 0 | 22 | 256 |
| Example 2 | Presence | 1 | 0 | 0 | 0 | 0.5 | 0 | 8 | 217 |
| Example 3 | Presence | 1 | 0 | 0 | 0 | 0 | 4 | 13 | 238 |
| Example 4 | Presence | 1 | 0 | 0 | 0 | 0.5 | 4 | 7.2 | 197 |
| Example 5 | Presence | 0 | 1 | 0 | 0 | 0.5 | 4 | 16 | 2311 |
| Example 6 | Presence | 0 | 0 | 1 | 0 | 0.5 | 4 | 9.1 | 199 |
| Example 7 | Presence | 0 | 0 | 0 | 1 | 0.5 | 4 | 18 | 222 |
| Comparative | Absence | 0 | 0 | 0 | 0 | 0 | 0 | 115 | 354 |
| Example 1 | | | | | | | | | |
| Comparative | Presence | 0 | 0 | 0 | 0 | 0 | 0 | 109 | 351 |
| Example 2 | | | | | | | | | |
| Cornparative | Absence | 0 | 0 | 0 | 0 | 0.5 | 0 | 76 | 279 |
| Example 3 | | | | | | | | | |
| Comparative | Presence | 0 | 0 | 0 | 0 | 0.5 | 0 | 71 | 266 |
| Example 4 | | | | | | | | | |
| Comparative | Absence | 0 | 0 | 0 | 0 | 0 | 4 | 88 | 325 |
| Example 5 | | | | | | | | | |
| Comparative | Presence | 0 | 0 | 0 | 0 | 0 | 4 | 84 | 310 |
| Example 6 | | | | | | | | | |
| Comparative | Absence | 0 | 0 | 0 | 0 | 0 | 0 | 36 | 263 |
| Example 7 | | | | | | | | | |

The official names of the respective additives abbreviated in Table 1 are presented below.

1,3-DOX: 1,3-dioxane

4-methyl-1,3-DOX: 4-methyl-1,3-dioxane (derivative of 1,3-dioxane)

Spiro compound: 3,9-divinyl-2,4,8,10-tetraoxaspiro[5.5]undecane (derivative of 1,3-dioxane)

1,4-DOX: 1,4-dioxane

VC: Vinylene carbonate

FEC: 4-fluoro-1,3-dioxolane-2-one

The following can be seen from Table 1.

In the batteries of Comparative Examples 1 and 2 in which both the first and second additives are not added to the electrolytic solution, the high temperature storage swelling is greatly severe and the degree of swelling hardly differs depending on whether or not the negative electrode binder has a mesh structure. It is presumed that this is because both the first and second additives are not added to the electrolytic solution, thus a solid electrolyte interphase is not formed on the positive electrode and the negative electrode, and a large amount of gas was generated at the positive electrode interface and the negative electrode interface mainly by the decomposition reaction of the electrolytic solution.

In the batteries of Comparative Examples 1 and 2, the increase in resistance after high temperature storage is also greatly severe and the degree of swelling hardly differs depending on whether or not the negative electrode binder has a mesh structure. It is presumed that this is mainly due to the inhibition of the charge transfer reaction by the increase in the distance between the positive and negative electrodes due to the generation of gas at the time of high temperature storage.

In the batteries of Comparative Examples 3 to 6 in which VC or FEC as a second additive is added to the electrolytic solution, high temperature storage swelling is suppressed as compared with that in the batteries of Comparative Examples 1 and 2 in which both the first and second additives are not added to the electrolytic solution. It is presumed that this is because the second additive is added to the electrolytic solution, thus a solid electrolyte interphase is formed on the negative electrode, the decomposition reaction of the electrolytic solution is suppressed at the negative electrode interface, and gas generation is suppressed. However, the magnitude of the effect of suppressing high temperature storage swelling hardly differs depending on whether or not the negative electrode binder has a mesh structure.

In the batteries of Comparative Examples 3 to 6, the increase in resistance after high temperature storage is suppressed as compared with that in the batteries of Comparative Examples 1 and 2. The magnitude of the effect of suppressing the increase in resistance differs depending on whether or not the negative electrode binder has a mesh structure, and the increase in resistance after high temperature storage is suppressed in each of the batteries of Comparative Examples 4 and 6 in which the negative electrode binder has a mesh structure as compared with that in the batteries of Comparative Examples 3 and 5 in which the negative electrode binder does not have a mesh structure. It is presumed that this is because the negative electrode active sites increase and a solid electrolyte interphase is effectively and ideally formed on the negative electrode when the negative electrode binder has a mesh structure.

In the batteries of Example 1 and Comparative Example 7 in which 1,3-dioxane as a first additive was added to the electrolytic solution, high temperature storage swelling and an increase in resistance are suppressed as compared with those in the batteries of Comparative Examples 3 to 6 in which the second additive is added to the electrolytic solution. The effect of suppressing the high temperature storage swelling and an increase in resistance greatly differs depending on whether or not the negative electrode binder has a mesh structure. Although the causal relationship between the mesh structure of the negative electrode binder and 1,3-dioxane that reacts on the positive electrode side is unknown, it is clear that high temperature storage swelling and an increase in resistance at the time of high temperature storage are suppressed by the combination of theses from the comparison of the evaluation results between Example 1 and Comparative Example 7.

In the batteries of Examples 2 and 3 in which 1,3-dioxane as a first additive and VC or FEC as a second additive are added to the electrolytic solution, high temperature storage swelling is suppressed as compared with that in the battery of Example 1 in which 1,3-dioxane as a first additive is added to the electrolytic solution. The details of the mechanism by which high temperature storage swelling is further suppressed when 1,3-dioxane and VC or FEC are combined with the mesh structure of the negative electrode binder are not clear, but it is presumed that this is because 1,3-dioxane that has a positive electrode interface protecting function suppresses the elution reaction of transition metals as well as the continuous radical reaction caused by the solvent decomposition at the negative electrode interface can be synergistically suppressed on both the positive and negative electrodes as the second additive forms a more stable solid electrolyte interphase on the mesh structure of the negative electrode binder.

In the batteries of Examples 2 and 3, the increase in resistance after high temperature storage is suppressed as compared with that in the battery of Example 1. The details of the mechanism by which the increase in resistance after high temperature storage is further suppressed when 1,3-dioxane and VC or FEC are combined with the mesh structure of the negative electrode binder are not clear, but it is presumed that this is because the interface protecting functions at both the positive and negative electrodes synergistically work similar to the above mechanism.

In the battery of Example 4 in which 1,3-dioxane as a first additive and VC and FEC as second additives are added to the electrolytic solution, high temperature storage swelling is suppressed as compared with that in the batteries of Examples 2 and 3 in which 1,3-dioxane as a first additive and VC or FEC as a second additive are added to the electrolytic solution. The details of the mechanism by which high temperature storage swelling is particularly suppressed when 1,3-dioxane and VC and FEC are combined with the mesh structure of the negative electrode binder are not clear, but it is presumed that the decomposition action on the negative electrode is different from that in the batteries of Examples 2 and 3 in the reaction field in which the negative electrode binder has a mesh structure and there is a large number of active sites and when VC and FEC that have different current and potential responsiveness from each other are concurrently used, and FEC reacts at a higher reduction potential, and thus the residual VC that did not fully react on the negative electrode can also secure the protecting function on the positive electrode side. Consequently, it is presumed that 1,3-dioxane that should react on the positive electrode side reacts more efficiently in a form closer to the ideal state than in the state in which the mesh structure of the negative electrode binder is not present.

In the battery of Example 4, the increase in resistance after high temperature storage is suppressed as compared with that in the batteries of Examples 2 and 3. The details of the mechanism by which the increase in resistance after high temperature storage is particularly suppressed when 1,3-dioxane and VC and FEC are combined with the mesh structure of the negative electrode binder are not clear, but it is presumed that the mechanism is similar to the above presumed mechanism, it is possible to form mixed solid electrolyte interphases with different ion permeability on the negative electrode, the reactivity of 1,3-dioxane that reacts in a high State of Charge (SOC) changes, and it is possible to decrease the charge transfer resistance at the positive electrode interface.

From these presumed mechanisms, it is considered that the suppression of gas generation at the time of high temperature storage and the suppression of resistance increase at the time of high temperature storage are achieved at the same time by concurrently using a mesh structure of the negative electrode binder which provides a large number of reaction active sites to the negative electrode, at least one of the first additive or the second additive (VC/FEC), more preferably both of these additives.

In the batteries of Examples 5 and 6 in which a derivative of 1,3-dioxane is added as the first additive as well, high temperature storage swelling and an increase in resistance after high temperature storage are suppressed similar to those in the battery of Example 4 in which 1,3-dioxane is added as the first additive.

In the battery of Example 7 in which 1,4-dioxane is added as the first additive as well, high temperature storage swelling and an increase in resistance after high temperature storage are suppressed similar to those in the battery of Example 4 in which 1,3-dioxane is added as the first additive.

However, in the battery of Example 4 in which 1,3-dioxane is added, high temperature storage swelling and an increase in resistance after high temperature storage are suppressed as compared with those in the battery of Example 7 in which 1,4-dioxane is added. The details of the mechanism by which high temperature storage swelling and an increase in resistance after high temperature storage are particularly suppressed when 1,3-dioxane is added are not clear, but it is presumed that 1,3-dioxane has a poorer electron density of the alkyl sandwiched between the ether structures as compared with 1,4-dioxane, thus 1,3-dioxane is highly reactive, that is, 1,3-dioxane is easily oxidized, and 1,3-dioxane more effectively exerts the effect than 1,4-dioxane.

The first to third embodiments of the present invention have been specifically described above, but the present invention is not limited to the above-described first to third embodiments, and various modifications can be made based on the technical idea of the present invention.

For example, the configurations, methods, steps, shapes, materials, numerical values and the like mentioned in the above-described first to third embodiments are merely examples, and configurations, methods, steps, shapes, materials, numerical values and the like different from these may be used, if necessary.

The configurations, methods, steps, shapes, materials, numerical values and the like of the above-described first to third embodiments can be combined with each other without departing from the gist of the present invention.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A non-aqueous electrolyte secondary battery comprising a positive electrode, a negative electrode, and an electrolyte, wherein
the negative electrode includes an active material and a binder having a mesh structure, and
the electrolyte includes at least one compound selected from the group consisting of a first cyclic ether having an ether structure at 1st and 3rd positions of a 6- or higher membered ring, a second cyclic ether having an ether structure at 1st and 4th positions of a 6- or higher membered ring, and derivatives of the first cyclic ether and the second cyclic ether.

2. The non-aqueous electrolyte secondary battery according to claim 1, wherein the electrolyte further includes a compound having LUMO energy of 0.60 eV or less.

3. The non-aqueous electrolyte secondary battery according to claim 1, wherein the electrolyte further includes at least one of a compound represented by following Formula (6) and a compound represented by following Formula (7): 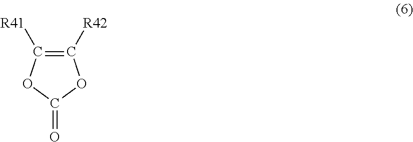
wherein each of R41 and R42 represents a hydrogen group or an alkyl group, 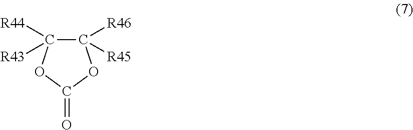
wherein each of R43 to R46 represents a hydrogen group, a halogen group, an alkyl group, or an alkyl halide group, and at least one of R43 to R46 represents a halogen group or an alkyl halide group.

4. The non-aqueous electrolyte secondary battery according to claim 1, wherein the electrolyte further includes at least one of a compound represented by following Formula (6) and a compound represented by following Formula (7): 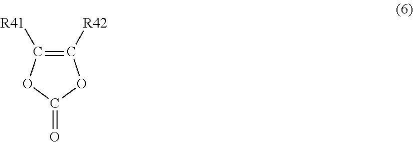
wherein each of R41 and R42 represents a hydrogen group or an alkyl group, 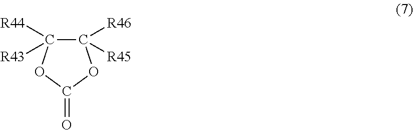
wherein each of R43 to R46 represents a hydrogen group, a halogen group, an alkyl group, or an alkyl halide group, and at least one of R43 to R46 represents a halogen group or an alkyl halide group.

5. The non-aqueous electrolyte secondary battery according to claim 1, wherein
the first cyclic ether includes 1,3-dioxane, 4-methyl-1,3-dioxane, or 3,9-divinyl-2,4,8,10-tetraoxaspiro[5.5]undecane, and
the second cyclic ether includes 1,4-dioxane.

6. The non-aqueous electrolyte secondary battery according to claim 2, wherein
the first cyclic ether includes 1,3-dioxane, 4-methyl-1,3-dioxane, or 3,9-divinyl-2,4,8,10-tetraoxaspiro[5.5]undecane, and
the second cyclic ether includes 1,4-dioxane.

7. The non-aqueous electrolyte secondary battery according to claim 3, wherein
the first cyclic ether includes 1,3-dioxane, 4-methyl-1,3-dioxane, or 3,9-divinyl-2,4,8,10-tetraoxaspiro[5.5]undecane, and
the second cyclic ether includes 1,4-dioxane.

8. The non-aqueous electrolyte secondary battery according to claim 1, wherein
the active material includes graphite, and
an intensity ratio (I(002)/I(110)) of an X-ray diffraction intensity I(002) of a (002) plane of the graphite to an X-ray diffraction intensity I(110) of a (110) plane of the graphite is 500 or more.

9. The non-aqueous electrolyte secondary battery according to claim 2, wherein
the active material includes graphite, and
an intensity ratio (I(002)/I(110)) of an X-ray diffraction intensity I(002) of a (002) plane of the graphite to an X-ray diffraction intensity I(110) of a (110) plane of the graphite is 500 or more.

10. The non-aqueous electrolyte secondary battery according to claim 3, wherein
the active material includes graphite, and
an intensity ratio (I(002)/I(110)) of an X-ray diffraction intensity I(002) of a (002) plane of the graphite to an X-ray diffraction intensity I(110) of a (110) plane of the graphite is 500 or more.

11. The non-aqueous electrolyte secondary battery according to claim 5, wherein
the active material includes graphite, and
an intensity ratio (I(002)/I(110)) of an X-ray diffraction intensity I(002) of a (002) plane of the graphite to an X-ray diffraction intensity I(110) of a (110) plane of the graphite is 500 or more.

12. The non-aqueous electrolyte secondary battery according to claim 1, wherein the non-aqueous electrolyte secondary battery includes a lithium ion battery.
